(19) United States
(12) Patent Application Publication
(10) Pub. No.: US 2024/0122528 A1
(43) Pub. Date: Apr. 18, 2024

(54) SEPSIS MONITORING

(71) Applicant: Welch Allyn, Inc., Skaneateles Falls, NY (US)

(72) Inventors: Chiew Yuan Chung, Singapore (SG); Christopher L. Long, Skaneateles Falls, NY (US); Craig M. Meyerson, Syracuse, NY (US); Rebecca Quilty-Koval, Baldwinsville, NY (US); Eugene G. Urrutia, Apex, NC (US); Swathi Utukuri, Skaneateles Falls, NY (US); Ching Yue Yeung, Fayetteville, NY (US)

(21) Appl. No.: 18/480,845

(22) Filed: Oct. 4, 2023

Related U.S. Application Data
(60) Provisional application No. 63/379,482, filed on Oct. 14, 2022.

(51) Int. Cl. A61B 5/00; A61B 5/0205; G16H 10/60; G16H 20/10

(52) U.S. Cl. CPC A61B 5/412; A61B 5/02055; A61B 5/7275; A61B 5/742; A61B 5/746; G16H 10/60; G16H 20/10; A61B 5/0816

(57) ABSTRACT

A system for monitoring sepsis in a healthcare facility. The system calculates sepsis scores for a patient using physiological parameter data captured by one or more sensors. The system generates a sepsis score trend based on the sepsis scores, and generates a predicted sepsis trend based on the sepsis scores and a treatment administered to the patient. The system displays the predicted sepsis trend over the sepsis score trend. The system can further display a summary including a sepsis status and a treatment status for a plurality of patients in the healthcare facility.

### BACKGROUND

Sepsis is a life-threatening condition that occurs when the body's response to infection causes injury to its own tissues and organs. Sepsis develops when a pathogen is released into the bloodstream that causes inflammation throughout the entire body.

In early stages, it is difficult to differentiate sepsis from other diseases because certain symptoms of sepsis, such as fever, increased heart rate, and breathing rate, mimic the symptoms of other diseases. The ability to detect sepsis at its earliest stages is critical because early sepsis is usually reversible with antibiotics, fluids, and other supportive medical interventions. However, as time progresses the risk of dying increases substantially.

### SUMMARY

In general terms, the present disclosure relates to monitoring sepsis in a healthcare facility. Various aspects are described in this disclosure, which include, but are not limited to, the following aspects.

One aspect relates to a system for monitoring sepsis in a healthcare facility, the system comprising: at least one processing device; and a memory device storing instructions which, when executed by the at least one processing device, cause the at least one processing device to: calculate sepsis scores for a patient using physiological parameter data captured by one or more sensors; generate a sepsis score trend based on the sepsis scores; generate a predicted sepsis trend based on the sepsis scores and a treatment administered to the patient; and display the predicted sepsis trend over the sepsis score trend.

Another aspect relates to a method for monitoring sepsis in a healthcare facility, the method comprising: calculating sepsis scores for a patient using physiological parameter data; generating a sepsis score trend based on the sepsis scores; generating a predicted sepsis trend based on the sepsis scores and a treatment administered to the patient; and displaying the predicted sepsis trend over the sepsis score trend.

Another aspect relates to a system for monitoring sepsis in a healthcare facility, the system comprising: at least one processing device; and a memory device storing instructions which, when executed by the at least one processing device, cause the at least one processing device to: acquire sepsis statuses from a plurality of monitor devices, each monitor device of the plurality of monitor devices monitoring a patient in the healthcare facility; acquire treatment statuses from an electronic medical record system, each treatment status associated with a patient monitored by the monitor devices; and display a user interface providing a summary having a sepsis status and a treatment status for each patent monitored by the monitor devices.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for monitoring sepsis.

FIG. 2 schematically illustrates an example of a monitor device in the system of FIG. 1, the monitor device shown as communicatively coupled to one or more sensors.

FIG. 3 illustrates an example of a user interface presented on a display device of the monitor device of FIG. 2.

FIG. 4 illustrates another example of a user interface presented on the display device of the monitor device of FIG. 2.

FIG. 5 schematically illustrates an example of a method of sepsis monitoring performed on the monitor device of FIG. 2.

FIG. 6 illustrates an example of a user interface presented on a display device of a status board in the system of FIG. 1.

FIG. 7 illustrates another example of a user interface presented on the display device of the status board in the system of FIG. 1.

FIG. 8 schematically illustrates an example of a method of sepsis monitoring performed on the status board in the system of FIG. 1.

FIG. 9 illustrates another example of a user interface presented on the display device of the status board in the system of FIG. 1.

### DETAILED DESCRIPTION

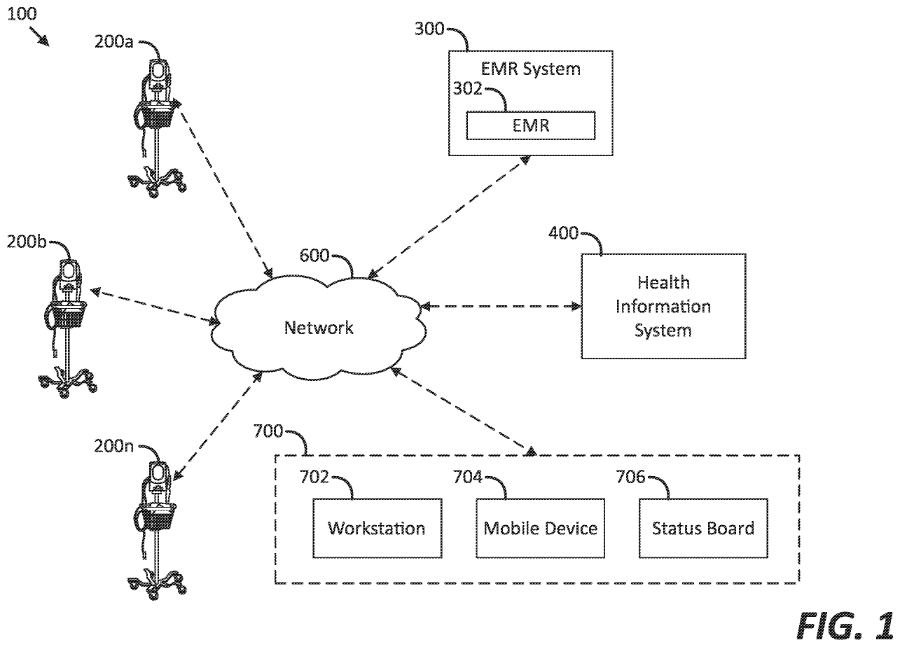

FIG. 1 schematically illustrates an example of a system 100 for monitoring sepsis. The system 100 can be implemented in a healthcare facility such as a hospital, a surgical center, a nursing home, a long term care facility, or similar type of facility. In some instances, the system 100 is implemented within a particular department, unit, or floor of a healthcare facility.

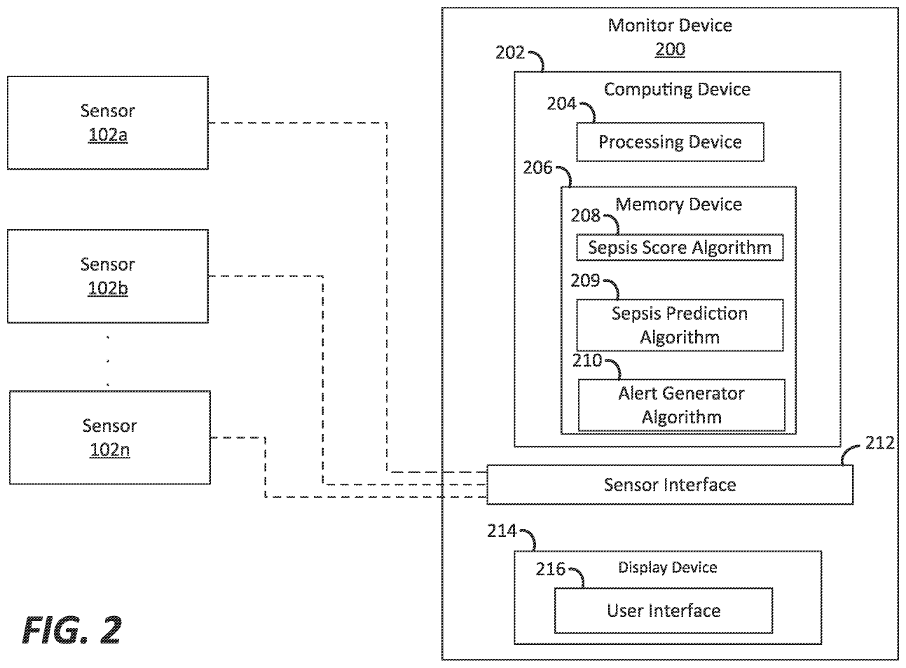

The system 100 includes one or more monitor devices 200a-200n connected to a network 600. Each of the one or more monitor devices 200a-200n obtains physiological parameter data from a patient admitted to the healthcare facility. The physiological parameter data acquired by the one or more monitor devices 200a-200n is used to calculate a sepsis score for each patient admitted to the healthcare facility. Also, a trend over time of the sepsis score for each patient is generated, and displayed on each monitor device 200a-200n. An example of a monitor device is shown in FIG. 2, and will be described in greater detail below.

As further shown in FIG. 1, an electronic medical record (EMR) system 300 (alternatively termed electronic health record (EHR) system) is connected to the network 600. The EMR system 300 stores the physiological parameter data acquired by the one or more monitor devices 200a-200n in an electronic medical record 302 (alternatively termed electronic health record) for each patient admitted in the healthcare facility. The EMR system 300 communicates with the one or more monitor devices 200a-200n over the network 600.

The system 100 further includes a health information system 400 that communicates with the one or more monitor devices 200a-200n and the EMR system 300 over the network 600. The health information system 400 centrally manages patient data to facilitate communications and coordination among healthcare providers in the healthcare facility. For example, the health information system 400 can collect and organize the patient data received over the network 600 from various devices and systems within the healthcare facility such as the one or more monitor devices 200a-200n and the EMR system 300.

The health information system 400 communicates the patient data to one or more additional devices 700 in the healthcare facility such as one or more workstations 702, mobile devices 704, and status boards 706. The health information system 400 can communicate the patient data to the one or more workstations 702, mobile devices 704, and status boards 706 over the network 600. Alternatively, the health information system 400 can communicate the patient data to the one or more workstations 702, mobile devices 704, and status boards 706 using a different network, and/or can communicate the patient data directly to the one or more workstations 702, mobile devices 704, and status boards 706 without using the network 600.

As an illustrative example, the one or more workstations 702 are part of a nurses station within the healthcare facility where nurses and other healthcare providers work and communicate with each other when not working directly with patients. The one or more mobile device 704 can include smartphones, tablet computers, and other portable computing devices that the nurses and other healthcare providers carry during their shifts within the healthcare facility. The one or more status boards 706 are monitors that are mounted to a wall or otherwise positioned within an area of the healthcare facility to centrally display patient data for all patients admitted to a particular department, unit, or floor within the healthcare facility.

The network 600 can include any type of wired or wireless communications, or any combinations thereof. Examples of wireless communications include broadband cellular network connections such as 4G or 5G. In some examples, wireless connections are accomplished using Wi-Fi, ultra-wideband (UWB), Bluetooth, radio frequency identification (RFID), and similar types of wireless connections. Wired connections can be established through Ethernet, and other standardized computer networking technologies. In some examples, the network 600 includes the Internet. In other examples, the network 600 is a local area network (LAN).

FIG. 2 schematically illustrates an example of a monitor device 200 communicatively coupled to one or more sensors 102a-102n that obtain physiological parameter data of a patient. The physiological parameter data captured by the one or more sensors 102a-102n can include, without limitation, respiration rate, blood pressure, heart rate, blood oxygen saturation (SpO₂), and body temperature. As will be described in more detail, the physiological parameter data is used to calculate sepsis scores for a patient, which are trended over time for sepsis monitoring.

As shown in FIG. 2, the monitor device 200 includes a computing device 202 having a processing device 204 and a memory device 206. The processing device 204 is an example of a processing unit such as a central processing unit (CPU), and in some instances can include one or more CPUs. In some examples, the processing device 204 can include one or more digital signal processors, field-programmable gate arrays, or other electronic circuits.

The memory device 206 operates to store data and instructions for execution by the processing device 204. In the example of FIG. 2, the memory device 206 stores a sepsis scoring algorithm 208 for calculating a sepsis score based on the physiological parameter data received from the one or more sensors 102a-102n. In some examples, the sepsis scoring algorithm 208 calculates a quick sequential organ failure assessment (qSOFA) score based on three factors: (1) low blood pressure (e.g., systolic blood pressure≤100 mmHg); (2) high respiration rate (≥22 breaths/min); and (3) altered mentation (e.g., Glasgow Coma Scale <15). Each factor is scored as 0 or 1 point based on whether the condition of the factor is satisfied or not, and the factors are summed together to calculate the qSOFA score.

As a further illustrative example, the sepsis scoring algorithm 208 calculates a systemic inflammatory response syndrome (SIRS) score based on four factors: (1) body temperature over 100.4 degrees Fahrenheit (38 degrees Celsius) or under 96.8 degrees F. (36 degrees C.); (2) heart rate greater than 90 beats per minute; (3) respiratory rate greater than 20 breaths per minute or partial pressure of CO2 less than 32 mmHg; and (4) leukocyte (white blood cell (WBC)) count greater than 12,000. Each factor is scored as 0 or 1 point based the condition of the factor being satisfied, and the factors are summed together to calculate the SIRS score.

In further examples, the sepsis scoring algorithm 208 calculates a custom score defined by a healthcare facility such as a hospital, a surgical center, a nursing home, a long term care facility, or similar type of facility, or a department, unit, or floor within the healthcare facility. The custom score can be based on a combination of factors such as blood pressure, respiration rate, mental state, body temperature, heart rate, white blood cell count, and the like.

As further shown in FIG. 2, the memory device 206 stores a sepsis prediction algorithm 209 for calculating a predicted sepsis score based on the sepsis scores of the patient and the treatment administered to the patient. As an illustrative example, the sepsis prediction algorithm 209 can predict changes in the individual factors that are used to calculate the sepsis score based on the treatment administered to the patient. For example, the sepsis prediction algorithm 209 can predict changes to one or more factors such as blood pressure, respiration rate, mental state, body temperature, heart rate, white blood cell count, and the like. The changes in these factors can then be used to calculate the predicted sepsis score.

The memory device 206 further stores an alert generator algorithm 210 that generates an alert, alarm, and/or notification based on the sepsis scores calculated by the sepsis scoring algorithm 208, or the predicted sepsis score calculated by the sepsis prediction algorithm 209.

The memory device 206 includes computer-readable media, which may include any media that can be accessed by the processing device 204. Examples of computer-readable media include computer-readable storage media that includes volatile and nonvolatile, removable and non-removable media implemented in any device configured to store information such as computer-readable instructions, data structures, program modules, or other data. Computer-readable storage media can include, but is not limited to, random access memory, read only memory, electrically erasable programmable read only memory, flash memory, and other memory technology, including any medium that can be used to store information that can be accessed by the monitor device 200. The computer-readable storage media is non-transitory.

Further examples of computer-readable media include computer-readable communication media that embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are within the scope of computer-readable media.

The monitor device 200 includes a sensor interface 212 that communicates with the one or more sensors 102a-102n. The sensor interface 212 can include both wired interfaces and wireless interfaces. For example, the one or more sensors 102a-102n can wirelessly connect to the sensor interface 212 through Wi-Fi, ultra-wideband (UWB), Bluetooth, and similar types of wireless connections. Alternatively, the one or more sensors 102a-102n can connect to the monitor device 200 through wired connections that plug into the sensor interface 212.

As shown in FIG. 2, the monitor device 200 includes a display device 214, which operates to display a user interface 216. In some examples, the display device 214 is a touchscreen such that the display device 214 operates as both a display device and a user input device. The monitor device 200 can also support physical buttons that operate to receive inputs from a healthcare provider to control operation of the monitor device 200 and enter data.

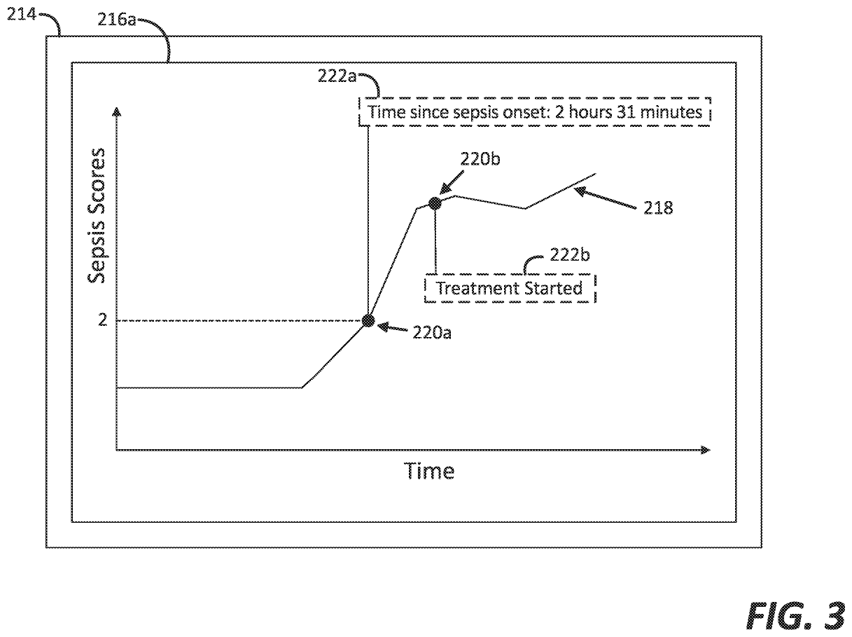

FIG. 3 illustrates an example of a user interface 216a displayed on the display device 214 of the monitor device 200. Similar user interfaces can be displayed on the one or more workstations 702, mobile devices 704, and status boards 706. The user interface 216a enhances the sepsis monitoring performed in the monitor device 200 by providing an output that is intuitive and easy to interpret by a healthcare provider to quickly understand a sepsis condition of a patient monitored by the monitor device 200, and thereby allowing the healthcare provider to more quickly take action, and improve patient healthcare and outcomes.

The user interface 216a includes a sepsis score trend 218. The sepsis score trend 218 is generated from the sepsis scores that are calculated by the sepsis scoring algorithm 208, which uses the physiological parameter data received from the one or more sensors 102a-102n as inputs. In the example of FIG. 2, the sepsis scoring algorithm 208 is stored on the memory device 206 of the monitor device 200 such that the sepsis scores are calculated on the monitor device 200. Alternatively, the sepsis scoring algorithm 208 can be stored on a memory of the health information system 400, such that the sepsis scores are calculated on the health information system 400, and are communicated over the network 600 to the monitor device 200.

In some examples, the sepsis scoring algorithm 208 utilizes artificial intelligence and/or machine learning algorithms to calculate the sepsis scores based on the physiological parameter data received from the one or more sensors 102a-102n. In some further examples, the monitor device 200 transmits the physiological parameter data received from the one or more sensors 102a-102n over the network 600 to the health information system 400, the health information system 400 performs the artificial intelligence and/or machine learning algorithms to calculate the sepsis scores based on the physiological parameter data, and the health information system 400 transmits the sepsis scores over the network 600 to the monitor device 200. By performing the artificial intelligence and/or machine learning algorithms on the health information system 400, the processing capacity needs of the computing device 202 are reduced, thus leading to more efficient use of the computing device 202 on the monitor device 200.

In some examples, the sepsis scores and the sepsis score trend 218 are saved in the electronic medical record 302 of the patient through transmission over the network 600 to the EMR system 300. In such examples, any of the one or more the monitor devices 200a-200n, the workstation 702, the mobile device 704, and the status board 706 can acquire the sepsis scores and the sepsis score trend 218 by accessing the electronic medical record 302 of the patient.

As shown in FIG. 3, the sepsis score trend 218 includes visual markers 220 that indicate events on the trend. For example, a first visual marker 220a identifies an onset of sepsis (e.g., when the patient is first identified as septic) on the sepsis score trend 218. A second visual marker 220b identifies a sepsis treatment start on the sepsis score trend 218. Additional visual markers can be displayed on the sepsis score trend 218 to identify additional clinical events.

Additionally, the sepsis score trend 218 can have a different visual appearance before and after a visual marker 220. For example, the sepsis score trend 218 can be drawn in different colors (including darker or lighter shades of color) and/or patterns to indicate changes before and after a visual marker 220. In the example of FIG. 3, the sepsis score trend 218 is drawn in a first color (e.g., grey) before the first visual marker 220a identifying the onset of sepsis, and is drawn in a second color (e.g., blue) after the first visual marker 220a identifying the onset of sepsis.

As shown in FIG. 3, the user interface 216a further provides one or more message boxes 222 each including a clinically relevant message describing a clinical event on the sepsis score trend 218. In FIG. 3, a first message box 222a includes a message “Time since sepsis onset: 2 hours 31 minutes”. A second message box 222b includes a message “Treatment started.”

The message boxes 222 provides further context for a healthcare provider to interpret the sepsis score trend 218 when viewing the user interface 216a on the monitor device 200.

The sepsis score trend 218 provides an output that is easily interpretable by a healthcare provider to validate whether treatments administered to the patient are improving the patient's sepsis score. For example, when the sepsis score trend 218 is downward (e.g., the sepsis score is decreasing) after the second visual marker 220b identifying the treatment start, this validates that the treatments administered to the patient are working such that no changes to the treatments are needed. Otherwise, when the sepsis score trend 218 is upward (e.g., the sepsis score is increasing) after the second visual marker 220b identifying the treatment start, this indicates a need to change the treatments to improve the patient's septic condition.

In some instances, when the sepsis score trend 218 is upward after the second visual marker 220b identifying the treatment start, such that the patient's condition is worsening, an alert is generated by the monitor device 200. For example, the alert generator algorithm 210 generates an alert or alarm when the monitor device 200 detects an increase in the sepsis score trend 218 after the treatment start. In some further examples, the alert generator algorithm 210 generates an alert or alarm when the monitor device 200 detects that the sepsis score trend 218 has not decreased during a predetermined period of time after the treatment start. In some further examples, the alert generator algorithm 210 generates an alert or alarm when the monitor device 200 detects that the sepsis score trend 218 has not improved (e.g., decreased) after 30 minutes.

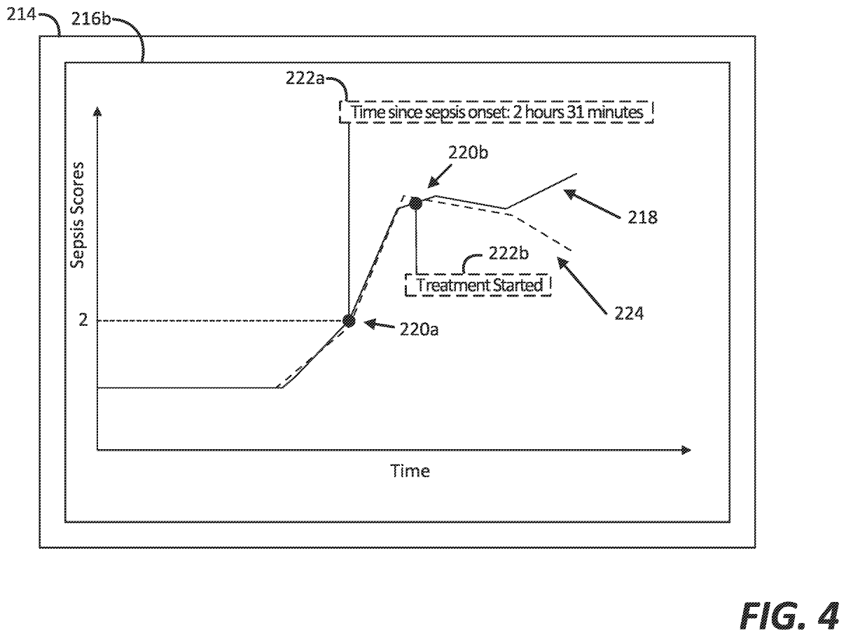

FIG. 4 illustrates another example of the user interface 216b displayed on the display device 214 of the monitor device 200. Similar user interfaces can be displayed on the one or more workstations 702, mobile devices 704, and status boards 706. The user interface 216b enhances the sepsis monitoring performed in the monitor device 200 by providing an output that is intuitive and easy to interpret by a healthcare provider to quickly understand a sepsis condition of a patient monitored by the monitor device 200, and thereby allowing the healthcare provider to more quickly take action, and improve patient healthcare and outcomes.

The user interface 216b in FIG. 4 shares many similar elements with the user interface 216a shown in FIG. 3. For example, the user interface 216b includes a sepsis score trend 218, visual markers 220a, 220b, and message boxes 222. Additionally, the user interface 216b includes a predicted sepsis trend 224 displayed together with the sepsis score trend 218. The predicted sepsis trend 224 is displayed as an overlay on the sepsis score trend 218 to visually indicate differences between the actual sepsis scores of the patient and the predicted sepsis scores of the patient after treatment is provided to the patient (e.g., after the second visual marker 220b).

The predicted sepsis trend 224 is drawn differently than the sepsis score trend 218 to accentuate and/or highlight the differences between the two trends. For example, the predicted sepsis trend 224 can be drawn in a different color (e.g., red) than the sepsis score trend 218 (e.g., blue). Additionally, and/or alternatively, the predicted sepsis trend 224 can be drawn in a different pattern (e.g., broken lines) than the sepsis score trend 218 (e.g., solid lines). Additional examples of how the predicted sepsis trend 224 can be drawn differently from the sepsis score trend 218 are possible such that these examples are provided by way of illustrative example.

The predicted sepsis trend 224 is calculated by the sepsis prediction algorithm 209, which uses the sepsis scores of the patient and the treatment administered to the patient as inputs to predict future sepsis scores for the patient over time after treatment starts. In some examples, the sepsis prediction algorithm 209 is trained using historical patient data that includes physiological parameter data of other patients with similar sepsis scores that were administered the same treatment as the patient monitored by the monitor device 200. In some examples, the sepsis prediction algorithm 209 is an artificial intelligence and/or machine learning algorithm.

As shown in the example of FIG. 2, the sepsis prediction algorithm 209 is stored on the memory device 206 of the monitor device 200 such that the predicted sepsis trend 224 is calculated on the monitor device 200. Alternatively, the sepsis prediction algorithm 209 can be stored on a memory of the health information system 400, such that the predicted sepsis trend 224 is calculated on the health information system 400, and is thereafter communicated by the health information system 400 over the network 600 for display on the monitor device 200.

As discussed above, in some examples, the sepsis prediction algorithm 209 utilizes artificial intelligence and/or machine learning algorithms to calculate the predicted sepsis trend 224. In such examples, the health information system 400 can perform the artificial intelligence and/or machine learning algorithms to calculate the predicted sepsis trend 224, and the health information system 400 transmits the predicted sepsis trend 224 over the network 600 for display on the monitor device 200. By performing the artificial intelligence and/or machine learning algorithms to calculate the predicted sepsis trend 224 on the health information system 400, the processing capacity needs of the computing device 202 of the monitor device 200 are reduced, thus leading to more efficient use of the computing device 202 of the monitor device 200.

In the illustrative example shown in FIG. 4, the predicted sepsis trend 224 after the second visual marker 220b (identifying treatment start) is trending downward, which is expected because the treatment administered to the patient should improve the patient's condition and lead to lower sepsis scores. However, in the example shown in FIG. 4, the sepsis score trend 218 is trending upward. This provides a visual output to the healthcare provider that the treatment administered to the patient is not working as intended because the sepsis score trend 218 does not match the predicted sepsis trend 224. In this particular example, the sepsis score trend 218 indicates that the treatment administered to the patient is not effective, and the patient's condition is in fact worsening. Thus, the sepsis score trend 218 when displayed together with the predicted sepsis trend 224 provides an output that is easily interpretable by a healthcare provider to validate whether treatments administered to the patient are working as intended.

In further examples, visual markers 220 are added to the sepsis score trend 218 when new treatments are administered to the patient. For example, when a first treatment is ineffective in lowering the sepsis scores of the patient, a second treatment can be administered to the patient. In such examples, a visual marker 220 is added to the sepsis score trend 218 to indicate the start time of the first treatment, and another visual marker 220 is added to the sepsis score trend 218 to indicate the start time of the second treatment. A visual marker 220 can be added to the sepsis score trend 218 each time a new treatment is administered to the patient.

In further examples, the predicted sepsis trend 224 is dynamically updated each time a new treatment is administered to the patient. For example, when a first treatment is ineffective in lowering the sepsis scores of the patient such that a second treatment is administered to the patient in an effort to improve the patient's condition, the predicted sepsis trend 224 is updated based on the second treatment administered to the patient. Thus, the predicted sepsis trend 224 can be continually updated when new treatments are administered to the patient.

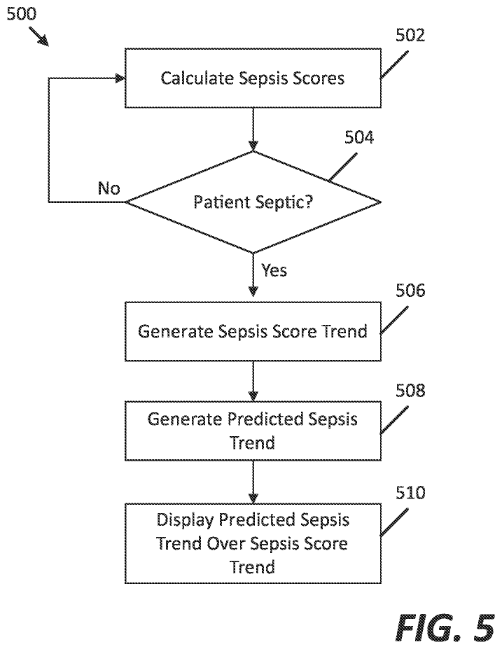

FIG. 5 schematically illustrates a method 500 of sepsis monitoring. In some examples, the method 500 is performed on the monitor device 200.

As shown in FIG. 5, the method 500 includes an operation 502 of calculating sepsis scores for the patient. The sepsis scores are calculated in operation 502 by the sepsis scoring algorithm 208 that uses the physiological parameter data acquired from the one or more sensors 102a-102b as inputs. In some examples, the sepsis scores are calculated in operation 502 utilizing artificial intelligence and/or machine learning algorithms. In some examples, the sepsis scores are calculated in operation 502 in predetermined intervals such as every 30 minutes.

The method 500 can include an operation 504 of determining whether the patient is septic based on the sepsis scores calculated in operation 502. For example, operation 504 can include determining whether the sepsis scores exceed a threshold for determining whether the patient is septic. When the patient is determined not to be septic (i.e., “No” in operation 504), the method 500 can return to operation 502 and continue to calculate the sepsis scores. When the patient is determined to be septic (i.e., “Yes” in operation 504), the method 500 can proceed to an operation 506 of generating the sepsis score trend 218. In some alternative examples, operation 504 is optional such that it is not included in the method 500.

Operation 506 includes generating the sepsis score trend 218 using the sepsis scores calculated in operation 502. In some examples, operation 506 can including drawing lines connecting the sepsis scores when the sepsis scores are calculated in predetermined intervals (e.g., every 30 minutes). In other examples, the sepsis scores are continuously calculated in operation 502 such that operation 506 does not including drawing lines connecting the sepsis scores. Operation 506 can include adding visual markers 220 such as a first visual marker 220a to identify on the sepsis score trend 218 when the patient was detected as septic in operation 504, and a second visual marker 220b to identify on the sepsis score trend 218 when a treatment is administered to the patient. Also, operation 506 can include adding one or more message boxes 222 providing clinically relevant message along with the sepsis score trend 218 such as to indicate an amount of time since the patient was detected as septic in operation 504.

Next, the method 500 includes an operation 508 of generating the predicted sepsis trend 224 based on the sepsis scores of the patient and the treatment administered to the patient. The predicted sepsis trend 224 is calculated in operation 508 by the sepsis prediction algorithm 209. In some examples, the sepsis prediction algorithm 209 is an artificial intelligence and/or machine learning algorithm that is trained using historical patient data.

Next, the method 500 includes an operation 510 of displaying the predicted sepsis trend 224 generated in operation 508 over the sepsis score trend 218 generated in operation 506. After completion of operation 510, the display of the predicted sepsis trend 224 over the sepsis score trend 218 can be provided in a user interface 216 such as the one shown in FIG. 4.

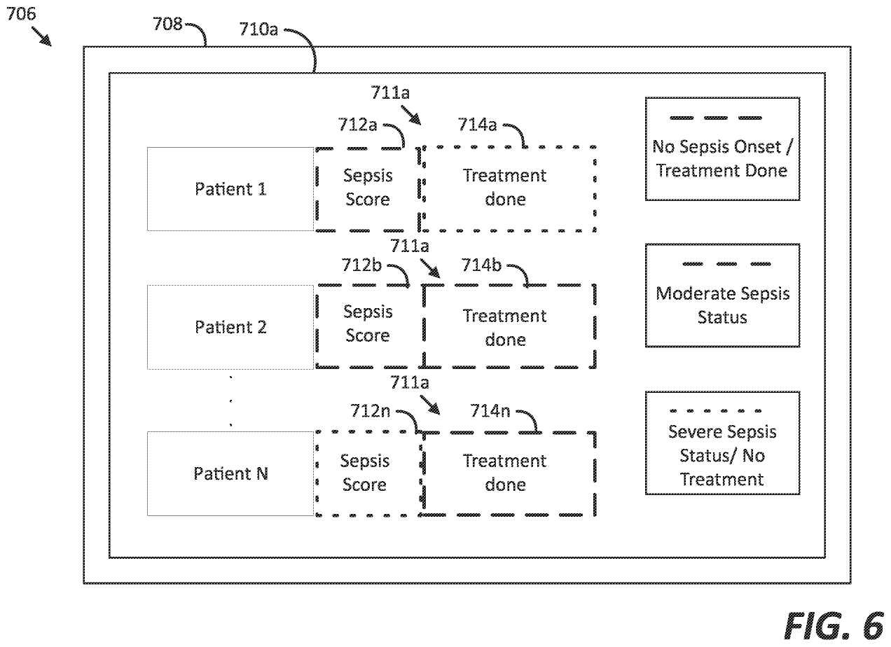

FIG. 6 illustrates an example of a user interface 710a presented on a display 708 of a status board 706. Similar user interfaces can also be displayed on the one or more monitor devices 200a-200n, workstations 702, and mobile devices 704. The user interface 710a summarizes sepsis statuses for patients admitted to the healthcare facility, or to a particular department, unit, or floor in the healthcare facility using data received over the network 600 from the one or more monitor devices 200a-200n and the EMR system 300. The user interface 710a displays the septic status of each patient and/or a quantity of high risk septic patients such that the user interface 710a can be used to determine how resources (including personnel staffing) within the healthcare facility, or a department, unit, or floor within the healthcare facility, should be allocated based on the sepsis statuses of the patients monitored by the monitor devices 200a-200n. Additionally, the user interface 710 can be used to determine which patients received appropriate sepsis treatment based on data received from the EMR system 300.

Referring back to FIG. 1, the status board 706 is communicatively connected to the one or more monitor devices 200a-200n via the network 600. Thus, the status board 706 shown in FIG. 6 can receive over the network 600 sepsis statuses of the patients monitored by the one or more monitor devices 200a-200n in near real-time. The status board 706 can receive over the network 600 the sepsis scores calculated by the one or more monitor devices 200a-200n using the sepsis scoring algorithm 208. Alternatively, the status board 706 can receive over the network 600 the sepsis statuses stored in the electronic medical records 302 of the patients in the EMR system 300. Additional examples of receiving the sepsis statuses are possible.

As further shown in FIG. 1, the status board 706 is communicatively connected to the EMR system 300 via the network 600. Thus, the status board 706 can receive over the network 600 the sepsis treatments that have been administered to the patients admitted to the department, unit, or floor of the healthcare facility based on data received from the EMR system 300.

In some examples, the status board 706 receives the sepsis statuses and sepsis treatments from the one or more monitor devices 200a-200n and the EMR system 300 using Health Level Seven (HL7) data communications. In some further examples, the sepsis statuses and treatments are auto populated on the status board 706 without user input.

Referring now to FIG. 6, the user interface 710a includes a list of patients (e.g., patient 1, patient 2 . . . patient N) admitted to the healthcare facility, or to a department, a unit, or a floor within the healthcare facility. For each patient in the list of patients, a summary 711a is provided that includes at least a sepsis status 712 and a sepsis treatment status 714.

The sepsis statuses 712 are displayed differently to visually distinguish severities of the sepsis statuses among the patients. For example, the sepsis statuses 712 can be color-coded to visually distinguish the severities of the sepsis statuses among the patients. In the example of FIG. 6, a sepsis status 712a for patient 1 can be colored yellow to indicate a moderate sepsis statis, a sepsis status 712b for patient 2 can be colored green to indicate no sepsis detected, and a sepsis status 712n for patient 1 can be colored red to indicate a severe sepsis status. Additional examples for visually distinguishing the sepsis statuses in the user interface 710a are possible.

The sepsis treatment statuses 714 are also displayed differently to distinguish whether sepsis treatments have been administered or not for each patient in the user interface 710a. For example, the sepsis treatment statuses 714 can be color-coded to visually distinguish whether sepsis treatment has been administered for each patient. In FIG. 6, a sepsis treatment status 714a for patient 1 can be colored red to indicate treatment has not been administered to patient 1 (who has a moderate sepsis status), and a sepsis treatment status 714n for patient N can be colored green to indicate that treatment has been administered to patient N (who has a severe sepsis status). A sepsis treatment status 714b for patient 2 can be colored green because the sepsis status 712b for patient 2 identifies no sepsis such that no treatment is necessary. Additional examples for visually distinguishing the sepsis treatment statuses are possible.

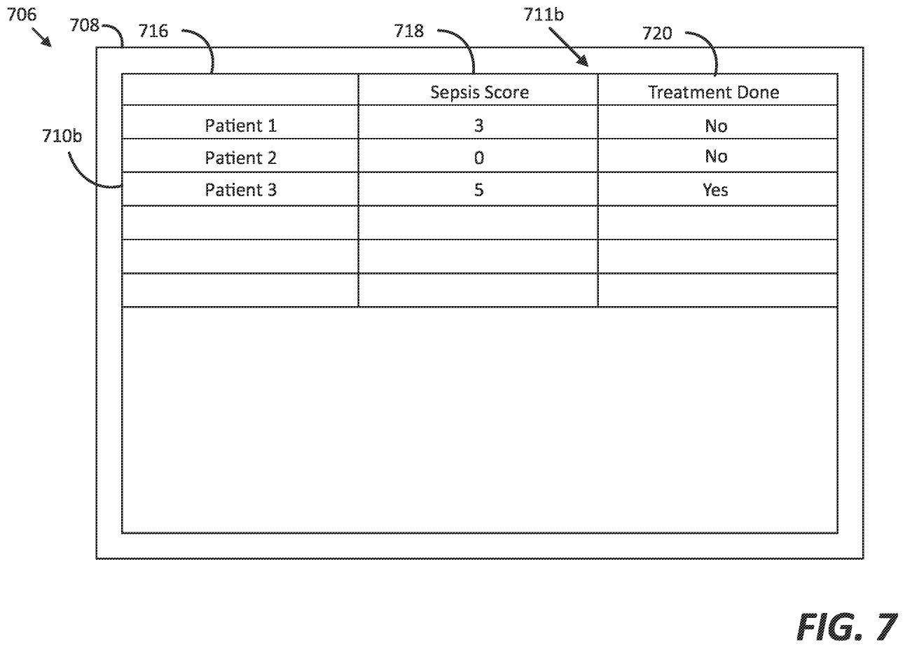

FIG. 7 illustrates another example of a user interface 710b presented on the display 708 of the status board 706. Similar user interfaces can also be displayed on the one or more workstations 702 and the one or more mobile devices 704. Like the user interface 710a described above, the user interface 710b shown in FIG. 7 provides a high-level view of sepsis statuses for patients admitted to the healthcare facility, or to a particular department, unit, or floor in the healthcare facility, using data received over the network 600 from the one or more monitor devices 200a-200n and the EMR system 300. The user interface 710b can be used to determine how resources within the healthcare facility, or a department, unit, or floor of the healthcare facility, should be allocated based on the sepsis statuses of the patients monitored by the one or more monitor devices 200a-200n. Also, the user interface 710b can be used to determine which patients received appropriate sepsis treatment based on data received from the EMR system 300.

As shown in FIG. 7, the user interface 710b includes a first column 716 listing the patients admitted to the healthcare facility, or to a particular department, a unit, or a floor in the healthcare facility. The user interface 710b further includes a summary 711b that includes a second column 718 listing sepsis scores for each patient listed in the first column 716, and a third column 720 listing sepsis treatment statuses for each patient listed in the first column 716. In the example of FIG. 7, the second column 718 provides that patient 1 has a sepsis score of 3 (e.g., a moderate sepsis status), and the third column 720 provides that patient 1 has not received sepsis treatment. The second column 718 further provides that patient 2 has a sepsis score of 0 (e.g., no onset of sepsis), and the third column 720 provides that patient 2 has not received sepsis treatment because treatment is not necessary given that patient 2 does not have sepsis. The second column 718 further provides that Patient 3 has a sepsis score of 5 (e.g., a severe sepsis status), and the third column 720 provides that patient 3 has received sepsis treatment.

The sepsis score values included in the second column 718 of the user interface 710b can be displayed differently to visually indicate a severity of the sepsis score for each patient admitted to the healthcare facility, or to a particular department, unit, or floor within the healthcare facility. For example, the sepsis score values can be color-coded. In the example of FIG. 7, the sepsis score of 3 for patient 1 can be colored yellow to indicate a moderate sepsis score, the sepsis score of 0 for patient 2 can be colored green to indicate no onset of sepsis, and the sepsis score of 5 for patient 3 can be colored red to indicate a severe sepsis score. Additional examples to visually distinguish the sepsis scores in the user interface 710b are possible.

Similarly, the sepsis treatment statuses included in the third column 720 of the user interface 710b can be displayed differently to visually indicate whether sepsis treatments have been administered or not for the patients admitted to the healthcare facility, or to a particular department, unit, or floor within the healthcare facility. For example, the sepsis treatment statuses can be color-coded. In the example of FIG. 7, the sepsis treatment status for patient 1 can be colored red to indicate that treatment has not been administered to patient 1. The sepsis treatment status for patient 3 can be colored green to indicate that treatment has been administered to patient 3. The sepsis treatment status for patient 2 is not colored because the sepsis score for patient 2 indicates no onset of sepsis such that no treatment is necessary. Further examples to distinguish the sepsis treatment statuses in the user interface 710b are possible.

As described above, the user interfaces 710a, 710b can also be displayed on the one or more mobile devices 704. In some examples, the one or more mobile devices 704 are each equipped with a clinical task management system such as the system described in U.S. patent application Ser. No. 16/674,735, which is herein incorporated by reference in its entirety. The clinical task management system when installed on the one or more mobile devices 704 facilitates communications between the healthcare providers in the healthcare facility. The communications in clinical task management systems installed on the one or more mobile devices 704 enable the healthcare providers to administer sepsis treatments in response to the treatment statuses included in the user interfaces 710a, 710b shown in FIGS. 6 and 7.

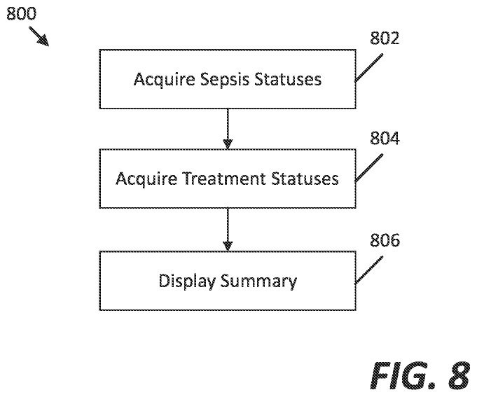

FIG. 8 schematically illustrates an example of a method 800 of sepsis monitoring. In certain examples, the method 800 is performed on the status board 706.

The method 800 includes an operation 802 of acquiring a sepsis status for each patient admitted to the healthcare facility, or to a department, unit, or floor within the healthcare facility. Operation 802 can include acquiring sepsis scores calculated by the one or more monitor devices 200a-200n using the sepsis scoring algorithm 208. Operation 802 can include acquiring the sepsis statuses over the network 600 from the one or more monitor devices 200a-200n. In some examples, operation 802 includes acquiring the sepsis statuses from the one or more monitor devices 200a-200n using HL7 data communications.

Next, the method 800 includes an operation 804 of acquiring sepsis treatment statuses of the patients admitted to the healthcare facility, or to a department, unit, or floor within the healthcare facility. Operation 804 can include acquiring the sepsis treatment statuses over the network 600 from the EMR system 300. In some examples, operation 804 includes acquiring the sepsis treatment statuses from the EMR system 300 using HL7 data communications.

Next, the method 800 includes an operation 806 of displaying sepsis summaries in a user interface, each sepsis summary including a sepsis status and a sepsis treatment status for a patent admitted to the healthcare facility, or to a department, a unit, or a floor in the healthcare facility. In some examples, operation 806 includes displaying the summaries 711a provided in the user interface 710a shown in FIG. 6. In other examples, operation 806 includes displaying the summaries 711b provided in the user interface 710b shown in FIG. 7.

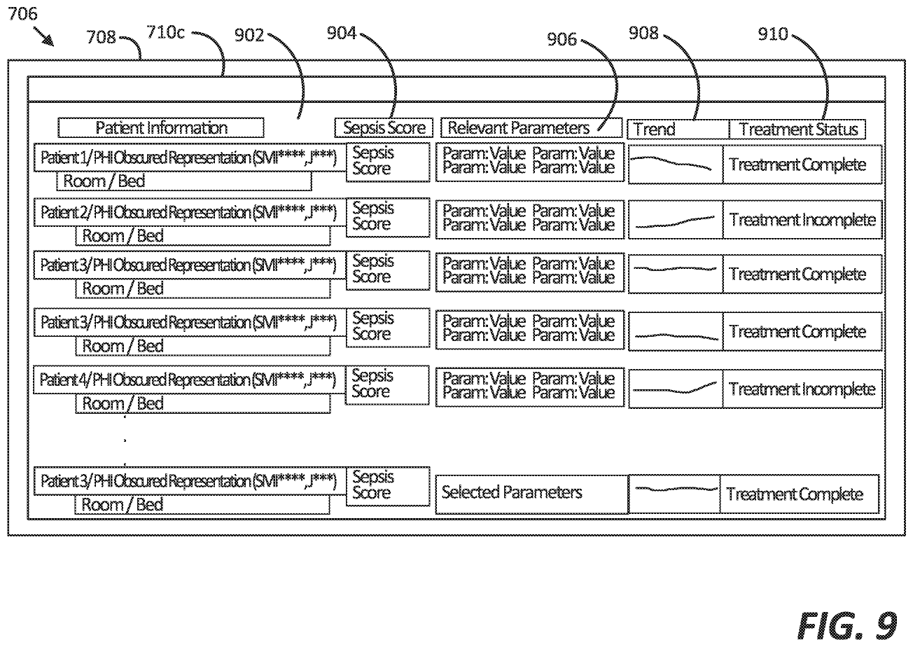

FIG. 9 illustrates another example of a user interface 710c presented on the display 708 of the status board 706. Similar user interfaces can also be displayed on the one or more workstations 702 and the one or more mobile devices 704. Like the user interfaces 710a, 710b described above, the user interface 710c provides a high-level view of sepsis statuses for patients admitted to the healthcare facility, or to a particular department, unit, or floor in the healthcare facility, using data received over the network 600 from the one or more monitor devices 200a-200n and the EMR system 300. The user interface 710c can be used to determine how resources within the healthcare facility, or a department, unit, or floor of the healthcare facility, should be allocated based on the sepsis statuses of the patients monitored by the one or more monitor devices 200a-200n. Also, the user interface 710c can be used to determine which patients received appropriate sepsis treatment based on data received from the EMR system 300.

As shown in FIG. 9, the user interface 710c lists in a first column 902 all patients admitted to the healthcare facility, or to a particular department, unit, or floor in the healthcare facility. For each patient, the first column can include their name, patient identification (ID) number, and/or patient room/bed number. Additional types of information for identifying the patients listed in the first column 902 are possible. Also, in some examples, the firs column can include less information for identifying each patient admitted to the healthcare facility.

The user interface 710c further includes a second column 904 that identifies the sepsis score for each patient listed in the first column 902. In accordance with the examples described above, the sepsis scores are acquired over the network 600 from the one or more monitor devices 200a-200n that utilize the physiological parameter data captured by the one or more sensors 102a-102n as inputs for the sepsis scoring algorithm 208 to calculate the sepsis scores as numerical values that represent a severity of the patient's sepsis condition.

The user interface 710c includes a third column 906 that provides the relevant physiological parameters that were used to calculate the sepsis scores identified for each patient in the second column 904. The third column 906 can be used to identify which of the physiological parameters contributed to a sepsis score being higher than normal for a patient.

The user interface 710c includes a fourth column 908 that includes the sepsis score trend 218 for each patient listed in the first column 902. As discussed above, the sepsis score trends 218 are generated from the sepsis scores that are calculated for each patient over a period of time. The sepsis score trends 218 can additionally be displayed on the one or more monitor devices 200a-200n that are monitoring the patients listed in the first column 902.

The user interface 710c includes a fifth column 910 that includes a sepsis treatment status for each patient listed in the first column 902. For example, the fifth column 910 can indicate whether sepsis treatment is complete or incomplete for each patient. In examples where a patient has a low sepsis score such that the patient is not at risk for sepsis, the fifth column 910 can indicate that the sepsis treatment status for the patient is complete since the sepsis treatment is not needed for the patient. Additional examples of sepsis treatment statuses are possible.

The various embodiments described above are provided by way of illustration only and should not be construed to be limiting in any way. Various modifications can be made to the embodiments described above without departing from the true spirit and scope of the disclosure.

What is claimed is:

1. A system for monitoring sepsis in a healthcare facility, the system comprising:
at least one processing device; and
a memory device storing instructions which, when executed by the at least one processing device, cause the at least one processing device to:
calculate sepsis scores for a patient using physiological parameter data captured by one or more sensors;
generate a sepsis score trend based on the sepsis scores;
generate a predicted sepsis trend based on the sepsis scores and a treatment administered to the patient; and
display the predicted sepsis trend over the sepsis score trend.

2. The system of claim 1, wherein the instructions, when executed by the at least one processing device, further cause the at least one processing device to:
add visual markers identifying clinical events on the sepsis score trend.

3. The system of claim 1, wherein the instructions, when executed by the at least one processing device, further cause the at least one processing device to:
add a first visual marker identifying an onset of sepsis on the sepsis score trend; and
add a second visual marker identifying a treatment start on the sepsis score trend.

4. The system of claim 1, wherein the instructions, when executed by the at least one processing device, further cause the at least one processing device to:
add one or more message boxes each describing a clinical event on the sepsis score trend.

5. The system of claim 1, wherein the instructions, when executed by the at least one processing device, further cause the at least one processing device to:
determine whether the patient is septic based on the sepsis scores.

6. The system of claim 1, wherein the instructions, when executed by the at least one processing device, further cause the at least one processing device to:
generate an alert when the sepsis score trend increases after a treatment start, or when the sepsis score trend does not decrease a predetermined period of time after the treatment start.

7. The system of claim 1, wherein the instructions, when executed by the at least one processing device, further cause the at least one processing device to:
display a summary including a sepsis status and a treatment status for each patient of a plurality of patients in the healthcare facility.

8. The system of claim 7, wherein the sepsis statuses are displayed differently to indicate a severity of the sepsis status for each patient, and the treatment statuses are displayed differently to indicate whether a treatment has been administered or not for each patient.

9. A method for monitoring sepsis in a healthcare facility, the method comprising:
calculating sepsis scores for a patient using physiological parameter data;
generating a sepsis score trend based on the sepsis scores;
generating a predicted sepsis trend based on the sepsis scores and a treatment administered to the patient; and
displaying the predicted sepsis trend over the sepsis score trend.

10. The method of claim 9, further comprising:
adding visual markers identifying clinical events on the sepsis score trend.

11. The method of claim 9, further comprising:
adding a first visual marker identifying an onset of sepsis on the sepsis score trend; and
adding a second visual marker identifying a treatment start on the sepsis score trend.

12. The method of claim 9, further comprising:
adding a message box describing a clinical event on the sepsis score trend.

13. The method of claim 9, further comprising:
generating an alert when the sepsis score trend increases after a treatment start, or when the sepsis score trend does not decrease a predetermined period of time after the treatment start.

14. The method of claim 9, further comprising:
displaying a summary having a sepsis status and a treatment status for each patient of a plurality of patients in the healthcare facility.

15. The method of claim 14, wherein the sepsis statuses are displayed differently to indicate a severity of the sepsis status for each patient, and the treatment statuses are displayed differently to indicate whether a sepsis treatment has been administered or not for each patient.

16. A system for monitoring sepsis in a healthcare facility, the system comprising:
at least one processing device; and
a memory device storing instructions which, when executed by the at least one processing device, cause the at least one processing device to:
acquire sepsis statuses from a plurality of monitor devices, each monitor device of the plurality of monitor devices monitoring a patient in the healthcare facility;
acquire treatment statuses from an electronic medical record system, each treatment status associated with a patient monitored by the monitor devices; and
display a user interface providing a summary having a sepsis status and a treatment status for each patent monitored by the monitor devices.

17. The system of claim 16, wherein the instructions, when executed by the at least one processing device, further cause the at least one processing device to:
display differently the sepsis statuses in the user interface to indicate a severity of the sepsis status for each patient; and
display differently the treatment statuses in the user interface to indicate whether sepsis treatments have been administered or not for each patient.

18. The system of claim 16, wherein the instructions, when executed by the at least one processing device, further cause the at least one processing device to:
calculate sepsis scores for each patient using physiological parameter data; and
generate a sepsis score trend for each patient based on the sepsis scores.

19. The system of claim 18, wherein the instructions, when executed by the at least one processing device, further cause the at least one processing device to:
generate a predicted sepsis trend for each patient based on the sepsis scores and a treatment administered to each patient; and
display the predicted sepsis trend over the sepsis score trend for each patient.

20. The system of claim 16, wherein the instructions, when executed by the at least one processing device, further cause the at least one processing device to:
generate an alert when the sepsis score trend for each patient increases after a treatment start, or when the sepsis score trend for each patient does not decrease a predetermined period of time after the treatment start.
